(19) United States
(12) Patent Application Publication
(10) Pub. No.: US 2021/0093642 A1
(43) Pub. Date: Apr. 1, 2021

(54) STABLE AQUEOUS PARENTERAL SOLUTIONS OF NONSTEROIDAL ANTI-INFLAMMATORY DRUGS (NSAIDs)

(71) Applicant: CADILA HEALTHCARE LIMITED, Ahmedabad (IN)

(72) Inventors: Kannan Essakimuthu MUTHAIYYAN, Ahmedabad (IN); Debjani Manoj SINGH, Ahmedabad (IN); Nirav Ishwarlal KHATRI, Ahmedabad (IN); Dhiraj Radheshyam SIKWAL, Ahmedabad (IN)

(73) Assignee: CADILA HEALTHCARE LIMITED, Ahmedabad (IN)

(21) Appl. No.: 17/032,442

(22) Filed: Sep. 25, 2020

(30) Foreign Application Priority Data
Sep. 26, 2019 (IN) 201921038952

(51) Int. Cl. A61K 31/5415; A61K 47/32; A61K 47/26; A61P 19/02; A61K 9/00

(52) U.S. Cl. CPC A61K 31/5415; A61K 47/32; A61K 9/0019; A61P 19/02; A61K 47/26

(57) ABSTRACT

The invention relates to stable, aqueous, parenteral solutions comprising one or more nonsteroidal anti-inflammatory drugs (NSAIDs) and polyvinylpyrrolidone. The present invention also relates to processes for processes preparing such solutions.

### FIELD OF THE INVENTION

The invention relates to stable, aqueous, parenteral solutions comprising one or more nonsteroidal anti-inflammatory drugs (NSAIDs) and polyvinylpyrrolidone. The present invention also relates to processes for preparing such solutions.

### BACKGROUND OF THE INVENTION

Meloxicam is a non steroidal anti-inflammatory drug (NSAID) of the oxicam class used to treat pain and inflammation in rheumatic diseases and osteoarthritis, via oral route (taken by mouth). It is recommended that it be used for as short a period as possible and at a low dose. Common side effects of meloxicam include abdominal pain, dizziness, swelling, headache, and a rash.

Metacam® (meloxicam) solution for injection was approved by the USFDA for use in the dogs for the control of pain and inflammation associated with osteoarthritis. Each mL of Metacam® product contains meloxicam 5 mg, alcohol 15%, glycofurol 10%, poloxamer 188 5%, sodium chloride 0.6%, glycine 0.5% and meglumine 0.3%, in water for injection, pH adjusted with sodium hydroxide and hydrochloric acid.

Loxicom® (meloxicam) solution for injection was approved by the USFDA for use in the cats for the control of postoperative pain and inflammation associated with orthopedic surgery, ovariohysterectomy and castration when administered prior to surgery. Each mL of Loxicom® product contains meloxicam 5 mg, alcohol 15%, glycofurol 10%, poloxamer 188 5%, sodium chloride glycine 0.5% and meglumine 0.3%, in water for injection, pH adjusted with sodium hydroxide and hydrochloric acid.

U.S. Publication No. 201010137292 A1 discloses a solution of meloxicam In combination with meglumine for administration by oral or parenteral route, comprising one or ore pharmaceutically acceptable excipients, characterized in that the solution is comprising N,N dimethylacetamide and propylene glycol. It discloses preferred solubilizers as propylene glycol and N,N-dimethylacetamide. It discloses preparation of meloxicam solution using meglumine and co-solvents, viz., N,N dimethyl acetamide and propylene glycol. It also discloses the usage of meglumine and high concentration of co-solvent to prepare the meloxicam solution, wherein the high concentration is more than 20% w/v.

U.S. Publication No. 2003101198.25 A1 (“the US'825 A1”) discloses aqueous solution containing meloxicam, meglumine, polyethylene glycol, polyoxyethylene-polyoxypropylene copolymer (poloxamer), ethanol, glycine and disodium EDTA, wherein the content of dissolved meloxicam salt is from 35 to 100 mg/mt. The formulations according to the US'825 A1 are suitable for treating animals, particularly domestic pets, working animals or farm animals.

U.S. Publication. No. 2014/0336163 A1 discloses composition of meloxicam containing hydroxypropyl-β-cyclodextrin, preservative (cresol etc.) and ethanol.

U.S. Publication No. 2005/0238280 A1 discloses use of meloxicam in veterinary medicine. It discloses meloxicam solution containing meglumine, macrogol (polyethylene glycol), poloxamer, ethanol and glycine. It also discloses meloxicam oily suspension.

U.S. Publication No. 2005/0245510 discloses use of meloxicam formulations in veterinary medicine. It discloses meloxicam solution containing meglumine, glycofurol poloxamer, ethanol and glycine. It also discloses meloxicam oily suspension.

U.S. Publication No 2013/0178467 discloses ,highly concentrated stable meloxicam solutions suitable for treating animals, preferably farm animals, and more particularly large farm animals.

International (PCT) Publication No. WO 2001/097813 (WO'811 discloses highly concentrated meloxicam solutions. It discloses meloxicam solution having concentration from 15 mg/mL or above, containing meglumine, polyethylene glycol, poloxamer, ethanol, glycine and disodium EDTA. The formulation according to WO'813 is suitable for treating animals, farm animals or large farm animals.

International (PCT) Publication No. WO 2008/062274 discloses solution of meloxicam containing ethyl alcohol and 2-Pyrollidone/N-methyl 2-Pyrollidone.

International (PCT) Publication No. WO 2019/037757 discloses injectable pharmaceutical compositions comprising meloxicam nanoparticles, a surface stabilizer and a sedimentation inhibitor. The stabilizers disclosed are polyvinylpyrrolidone, polyvinyl alcohol, hydroxypropylmethylcellulose, Tween 80, poloxamer, polyethylene glycol stearate, lecithin, sodium deoxycholate, sodium cholate, sodium dodecyl sulfate or sodium lauryl sulfate. The sedimentation inhibitors disclosed are glycerin, propylene glycol, polyethylene glycol, albumin, hydroxyethyl starch, sodium carboxymethyl cellulose or hydroxypropyl-62 -cyclodextrin. It also discloses an injectable pharmaceutical composition comprising meloxicam nanoparticles, polyvinylpyrrolidone, sodium deoxycholate, sedimentation inhibitor, and water, wherein the meloxicam nanoparticles have an average particle size of less than 500 nm, preferably less than 200 nm.

SUN Ying-hua et al.; Journal of Shenyang Pharmaceutical University; 2004-02, discloses utilization of 2-hydroxpropyl-β-cyclodextrin as the solubilizer for meloxicam.

Chinese Publication No, 1493292 A discloses a liquid preparation of meloxicam prepared from meloxicam or medical salt and water soluble cyclodextrin derivative HP-beta-CD used a solubilizer and a stabilizer.

ZHAO Jun et al.; Journal of China Pharmaceutical University, “Study on the solubilization of meloxicam in meglumine aqueous solution”, 2003, Vol. 34, No. 5, abstract, discloses that meloxicam is a hydrophobic drug and difficult to dissolve in aqueous solution. Meglumine can increase the solubility of meloxicam n aqueous solution.

There still exists a need for a storage stable solution which enables safe and efficacious administration of solution comprising meloxicam which is suitable for administration via subcutaneous, intravenous or intramuscular route.

### SUMMARY OF HE INVENTION

one general aspect, the present invention provides stable, aqueous, parenteral ,solution comprising a nonsteroidal anti-inflammatory drug and polyvinylpyrrolidone (PVP).

In one general aspect, the present invention provides a stable, aqueous, parenteral solution comprising meloxicam and polyvinylpyrrolidone (PVP).

In another general aspect, the present invention provides a process for preparing the parenteral solution comprising meloxicam and polyvinylpyrrolidone.

In another general aspect, the present invention provides a method of treating acute pain, osteoarthritis, rheumatoid arthritis and juvenile rheumatoid arthritis pauciarticular and polyarticular course, the method comprising administering to a human being in need thereof, the parenteral solution of the present invention.

The details of one or more embodiments of the invention are set forth in the description below. Other features, objects and advantages of the invention will be apparent fro the description,

### DETAILED DESCRIPTION OF THE INVENTION

The present invention provides a stable, aqueous, parenteral solution comprising one or more nonsteroidal anti-inflammatory drug and one or more pharmaceutically acceptable excipients.

Suitable drug for the composition of the invention may have low water solubility or may be water insoluble. Examples of suitable nonsteroidal anti-inflammatory drugs may include one or more of meloxicam, ampiroxicam, piroxicam, tenoxicam, droxicam, lornoxicam, isoxicam, etc.

The aqueous parenteral solution is in the far clear solution comprising meloxicam and polyvinylpyrrolidone,

The storage stable aqueous parenteral solution may be administered via parenteral route, for example, intramuscular (intradeltoid or intragluteal), subcutaneous, intravenous (IV infusion or IV bolus route).

The term “about” as used herein, refers to encompass +/−20%, 15%, 10%, 5%, 2%, 1%, 0.5%, or (25% of the numerical value of the number with which it is being used.

The term “between” as used herein for the purpose of defining range inclusive of the lower and upper number of the range.

The term “storage stable” as used herein, refers to any composition comprising a drug having sufficient physical and chemical stability to allow storage at a convenient temperature, such as between about 0° C. and about 40° C., for a commercially reasonable period of time. The term “physical stability” refers to maintenance of color or colorless state, dissolved oxygen level head space, oxygen level, and particulate matter size. The term “chemical stability” relates to formation of drug-related impurities in terms of total impurities, known impurities and single maximum unknown impurity, up to allowed limits by the Regulatory Agency. For pharmaceutical products, stability is required for commercially relevant time after manufacturing, such as for about 1, 3, 6, 12, 18, 24 or 36 months, during which a product is kept in its original packaging under specified storage conditions. The term composition includes the solution prepared in accordance with the present invention.

The term “stable” as used herein, refers to any composition comprising a drug having sufficient physical and chemical stability at the time of manufacturing of the composition.

The term “controlled room temperature” as used herein, refers to the temperature between 20° C. and 25° C.

The term “Tₘₐₓ” used herein, unless and otherwise specifically mentioned, means time to achieve maximum concentration of the drug in plasma, achieved after administration of the product (unit for example: minutes).

The term “Cₘₐₓ” as used herein, unless and otherwise specifically mentioned, means maximum concentration of drug in plasma, achieved after administration of the product (unit for example: ng/mL).

The term “AUG” as used herein, unless and otherwise specifically mentioned, means area under the curve for a plot of concentration of drug in plasma vs. time (unit for example: ng/mL).

As used herein, the term ‘clear solution’ means a solution which does not contain any visible particulate matter, solid particle, liposome or nanoparticles. The dear solution provides % transmittance, when measured at 650 nm, not less than 97 example, not less than 98%, less than 99%, not less than 99.5%, not less than 99.6%, not less than 99.7% or not less than 99.8%.

In one embodiment, the present invention provides a stable parenteral solution comprising meloxicam, polyvinylpyrrolidone and water.

In another embodiment, the present invention provides a stable aqueous solution comprising meloxicam, polyvinylpyrrolidone, polysorbate 80 and water for parenteral administration.

In another embodiment the present invention provides a storage stable aqueous parenteral solution comprising meloxicam, polyvinylpyrrolidone, polysorbate 80 and water, wherein the solution is storage stable after storage for 12 months at controlled room temperature.

The stable aqueous parenteral solution may comprise meloxicam in a concentration between of about 1 mg/mL and about 50 mg/mL, for example, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 20 mg/mL, about 25 mg/mL, about 30 mg mL, about 35 mg/mL, about 40 mg/mL, about 45 mg/mL or about 50 mg/mL.

The stable aqueous parenteral solution may comprise polyvinylpyrrolidone in a concentration between of about 5 mg/mL and about 200 mg/mL, for example, about 10 mg/mL, about 30 mg/mL, about 40 mg/mL, about 45 mg/mL, about 50 mg/mL, about 60 mg/mL, about 70 mg/mL, about 80 mg/mL, about 90 mg/mL, about 100 mg/mL, about 125 mg/mL, about 150 mg/mL or about 175 mg/mL.

In another embodiment, the stable aqueous parenteral solution may comprise meloxicam in a concentration of 10 mg/mL or 15 mg/mL, wherein the solution does not require any dilution step before administration, which remains clear (free of any crystals/precipitates) after storage for at least 6 months when stored at controlled room temperature or at 2° C.-8° C. temperature. The solution is ready to use for administration via intravenous, subcutaneous or intramuscular route.

In another embodiment, the present invention provides a kit comprising an auto-injector which contains a pre-filled syringe (a pre-filled syringe assembled/placed in an auto-injector), wherein the pre-filled syringe (PFS) contains meloxicam solution in a therapeutically effective amount. The auto-injector provides convenience to the patient for the self-administration.

In another embodiment, the present invention provides a stable parenteral solution comprising meloxicam, polyvinylpyrrolidone and water, wherein the solution is supplied/provided in a suitable packaging material, for example, in a glass vial, in a glass ampoule, in a pre-filled syringe, in a glass bottle, in a plastic bottle, in a plastic bag, etc. The pre-filled syringe contains various constituent parts, for example, a sterile dear USP Type-I siliconized glass syringe barrel (1 mL, cut flange with a gauge (29 size), hypodermic needle (½ inch) fitted with rigid needle shield and laminated bromobutyl plunger stopper for the barrel.

In another embodiment, the present invention provides a kit comprising an auto-injector which contains a pre-filled syringe (a pre-filled syringe assembled/placed in the auto-injector), wherein the pre-filled syringe contains a solution comprising meloxicam, polyvinylpyrrolidone and water.

In another embodiment, the present invention provides a kit comprising (a) a pre-filled syringe containing a solution comprising meloxicam, polyvinylpyrrolidone and water and (b) an auto-injector device. The kit is suitable to administer the solution via subcutaneous route.

The autoinjector may be integrated with a needle stick protection feature and holds a pre-filled syringe containing a single dose, whereby the entire deliverable volume is expelled.

In another embodiment, the present invention provides a single-dose pre-filled syringe with an auto-injector, wherein the pre-filled syringe contains solution comprising meloxicam, polyvinylpyrrolidone and water, wherein the pre-filled syringe with an auto-injector is suitable to administer the effective amount of meloxicam dose via subcutaneous route for the acute treatment of pain.

In another embodiment, the present invention provides a meloxicam injection in a single-dose pre-filled syringe with an auto-injector.

In another embodiment, the present invention provides a meloxicam injection in a single-dose pre-filled syringe with an auto-injector, wherein the meloxicam is present in a therapeutically effective dose amount from about 7.5 mg to about 60 mg, for example 10 mg, 15 mg, 20 mg, 25 mg, 30 mg, 35 mg, 40 mg, 45 mg, 50 mg or 55 mg.

In one embodiment, the present invention provides a storage stable aqueous parenteral solution comprising meloxicam and one or more pharmaceutically acceptable excipients, wherein the solution retains at least 95% of the meloxicam.

The storage stable aqueous parenteral solution retains at least 95% of the meloxicam (% assay) after storage for at least 1 month, for example, 2 months, 3 months, 6 months, 12 months, 18 months, or 24 months at controlled room temperature (CRT).

The storage stable aqueous parenteral solution retains at least 95% of the meloxicam (% assay) after storage for at least 1 month, for example, 2 months, 3 months, 6 months, 12 months, 18 months, or 24 months at 25° C. temperature and 60% relative humidity (% RH).

The storage stable aqueous parenteral solution retains at least 95% of the meloxicam (% assay) after storage for at least 1 month, for example, 2 months, 3 months, 6 months, 12 months, 18 months, or 24 months at 40° C. temperature and 75% RH.

In another embodiment, the stable aqueous parenteral solution comprising meloxicam is dear (free of any crystals/precipitates) by visual, inspection after storage for at least 1 month, for example, 2 months, 3 months, 6 months, 12 months, 18 months, or 24 months, at controlled room temperature. The solution of the present invention provides value of absorbance not more than 1, for example, not more then 0.75, 0.5, 0.4, 0.3, 0.2, 0.1 or 0.05 and value of % transmittance not less than 90%, for example, not less than 95%, 96%, 97%, 98%, 99%, 99.5% or 99.9%.

In another embodiment, the stable aqueous parenteral solution comprising meloxicam does not contain 4-Hydroxy-2-methyl-2H-1,2-benzothiazine-3-carboxylic acid ethyl ester 1, 1-dioxide. (Impurity A) more than 0.5%, for example, 0.4%, 0.3%, 0.2%, 0.1% or 0.05%, by weight of meloxicam, as measured by HPLC.

In another embodiment, the stable aqueous parenteral solution comprising meloxicam does not contain 2-Amino-5-methyl-thiazole (Impurity B) more than 0.5%, for example, 0.4%, 0.3%, 0.2%, 0.1% or 0.05%, by weight of meloxicam, as measured by HPLC.

In another embodiment, the stable aqueous parenteral solution comprising meloxicam does not contain N-(3,5-dimethylthiazol-2(3H)-ylidene)-4-hydroxy-2-methyt-2H-benzo[e][1,2]thiazine-3-carboxamicle 1,1-dioxide (Impurity E) more than 05%, for example, 0.4%, 0.3%, 0.2%, 0.1% or 0.05%, by weight of meloxicam, as measured by HPLC.

In another embodiment, the stable aqueous parenteral solution comprising meloxicam does not contain N-(3-Ethyl-5-methylthiazol-2(3H)-ylidene)-4-hydroxy-2-methyl-2H-benzo[e][1,2]thiazine-3-carboxamide 1-dioxide (Ethyl meloxicam impurity) more than 0.5%, for example, 0.4%, 0.%, 0.2%, 0.1% or 0.05%, by weight of meloxicam, as measured by HPLC.

In another embodiment, the stable aqueous parenteral solution comprising meloxicam does not contain Methyl4-hydroxy-2-methyl-2H-1.2-benzothiazine-3-carboxylate 1,1-Dioxide (Impurity-D) more than 0.5%, for example, 0.4%, 0.3%, 0.2%, 0.1% or 0.05%, by weight of meloxicam, as measured by HPLC.

In another embodiment, the stable aqueous parenteral solution comprising meloxicam does not contain 2-(diazenyisulfonyi)-N-methylaniline oxide (Meloxicam diazenyl sulfonyl Impurity) more than 1%, for example, 0.8%, 0.5%, 0.4%, 0.3%, 0.2%, 0.1% or 0.055 by weight of meloxicam, as measured by HPLC.

In another embodiment of the invention, there is provided a parenteral solution comprising meloxicam, wherein the solution does not contain single maximum unknown impurity more than 1% and/or does not contain total impurities more than 3% (for example, not more than 2%, 1%, or 0.5%) after storage for more than 2 months, for example, for 3 months, for 6 months, for 12 months, for 18 months, for 24 months or for 36 months when stored at (i) 2° C.-8° C. temperature or at (ii) 25±2° C. temperature and 60±5% RH or at (iii) 40±2° C. and 75±5% RH. The parenteral solution comprising meloxicam of the present invention does not form any precipitate and remains physically stable after storage for more than 2 months, for example, for 3 months, for 6 months, for 12 months, for 18 months, for 24 months or for 36 months when stored at 2° C.-8° C. temperature or at 25±2° C. temperature and 60±5% RH.

In another embodiment, the stable aqueous parenteral solution comprising meloxicam has a viscosity value between of about 1 cP (centipoise) and 5 cP, for example, 1.5 cP, 2 cP, 2.5 cP, 3 cP, 3.5 cP, 4 cP or 4.5 cP.

In another embodiment, the stable aqueous parenteral solution comprising, meloxicam has an osmolality value of between about 200 mOsm and about 600 mOsm, for example, about 250 mOsm, about 300 mOsm, about 350 mOsm, about 400 mOsm, about 450 mOsm, about 500 mOsm or about 550 mOsm.

In another embodiment, the stable aqueous parenteral solution comprising meloxicam has a pH of between about 7 and about 10, for example, about 7.5, about 8, about 8.5, about 9 or about 9.5.

The suitable pharmaceutically acceptable excipients for the solution of the present invention may include one or more pharmaceutically acceptable solvents, solubilizers, stabilizers, preservatives, antioxidants, surfactants, buffering agents, nucleation inhibitors, pH adjusting agents and isotonicity adjusting agents.

Examples of suitable pharmaceutically acceptable solvents may include, but not limited to, water for injection, and the like.

Examples of suitable pharmaceutically acceptable solubilizers may include, but not limited to benzyl benzoate, castor oil, cottonseed oil, N, N dimethylacetamide, dehydrated ethanol, glycerol, N-methyl-2-pyrrolidone, diethanolamine, L-arginine, peanut oil, poppyseed oil, safflower oil, sesame oil, soybean oil, vegetable oil, or any combination thereof.

Examples of suitable pharmaceutically acceptable stabilizers may include may include, but not limited to aminoethyl sultonic acid, L-arginine, butylhydroxyanisol, polyvinylpyrrolidone, L-cysteine, cysteine hydrochloride, diethanolamine diethylenetriaminepentaacetic acid, ferric chloride, inositol, D,L-methionine, or any combination thereof.

Examples of suitble pharmaceutically acceptable preservatives may include, but not limited to, chlorobutanol, benzalkonium chloride, methyl paraben, propyl paraben, benzoic acid, sodium benzoate, sorbic acid, benzethonium chloride, cetyl pyridinium chloride, benzyl bromide, benzyl alcohol, phenylmercury nitrate, phenylmercury acetate, thiomersal, merthiolate, chlorhexidine, phenylethyl alcohol, quaternary ammonium chloride, sodium benzoate, sodium propionate, or any combination thereof.

Examples of suitable pharmaceutically acceptable anti-oxidants may include, but not limited to, butylateci hydroxytoluene (BHT), butylated hydroxyanisole (BHA), propyl gallate, monothioglycerol, ascorbic acid, sodium ascorbate, erythorbic acid, potassium metabisulfite, sodium metabisulfite, propionic acid, sodium formaldehyde sulphoxylate, reduced glutathione, thiourea, cysteine, n-acetylcysteine, methionine, sodium sulfite, sodium bisulfate, alkyl gallate, including propyl gallate, vitamin E, or other tocopherol analogs, including tocopherol acetate or TPGS, or any combination thereof.

Examples of suitable pharmaceutically acceptable surfactants may include, but not limited to, amphoteric, non-ionic, cationic or anionic molecules. Suitable surfactants may include, but not limited to, polysorbate 80 (e.g. tween 80 etc.), poloxamer (poloxamer 188), sodium lauryl sulfate, lauryl dimethyl amine oxide, docusate sodium, cetyl trimethyl ammonium bromide (CTAB), polyvinyl alcohol, polyethoxylated alcohols, polyoxyethylene sorbitan, octoxynol, polyoxyl lauryl ether, Brij® surfactants (polyoxyethylene vegetable-based fatty ethers derived from lauryl, cetyl, stearyl and oleyl alcohols), bile salts (such as sodium deoxycholate and sodium cholate), polyoxyl castor oil, nonylphenol ethoxylate, lecithin, polyoxyethylene surfactants, phospholipids such as dimyristoylphosphatidyl glycerol (DMPG), disteroylphosphatidylethanolamine (DSPE), 1,2-Distearoyl-phosphatidylethanolarnine-methyl-polyethylene glycol conjugate (DSPE-mPEG) monoakanolamine condensates, polyoxyethylerie fatty acid amides, quaternary ammonium salts, polyoxyethylene alkyl and alicyclic amines, polyoxyethylene, sorbitan monolaurate and stearate, Solutol® (ethylene oxide/12-hydroxy stearic acid), tyloxapol, or any combination thereof.

The stable aqueous parenteral solution of the present invention may comprise polysorbate 80 in a concentration of between about 1 mg mL and about 100 mg/mL, for example, about 2 mg/mL, about 5 mg/mL, about 7 mg/mL, about 10 mg/mL, about 20 mg/mL, about 40 mg/mL, about 50 mg/mL, about 60 mg/mL or about 80 mg/mL.

Examples of suitable pharmaceutically acceptable buffering agents may include, but not limited to, acetate (e.g. sodium acetate etc.), citrate (e.g. citric acid/sodium citrate etc.), phosphate (e.g. monobasic sodium phosphate, dibasic sodium phosphate etc.), carbonate, glycine, borate (boric acid/potassium chloride), or any combination thereof.

The stable aqueous parenteral solution of the present invention may comprise glycine in a concentration of between about 1 mg/mL and about 10 mg/mL, for example, about 2 mg/mL, about 4 mg/mL, about 5 mg/mL, about 6 mg mL or about 8 mg/mL.

Examples of suitable pharmaceutically acceptable nucleation inhibitors may include, but not limited to, polyvinylpyrrolidone, crospovidone, hydroxypropylmethyl cellulose (HPMC), poloxamers, polysorbate, phospholipids such as dimyristoylphosphatidyl glycerol (DMPG), disteroylphosphatidylethanolamine (DSPE), 1,2-Distearoyl-phosphatidylethanolamine-methyl-polyethylene glycol conjugate (DSPE-mPEG), or any combination thereof. In one embodiment, polyvinylpyrrolidone may be PVP K12, PVP K17, PVP K25, PVP K30, PVPK 40 or PVP K90.

Examples of suitable pharmaceutical y acceptable pH adjusting agents may include, but not limited to, sodium hydroxide, hydrochloric acid, boric acid, citric acid, acetic acid, phosphoric acid, succinic acid, potassium hydroxide, ammonium hydroxide, magnesium oxide, calcium carbonate, magnesium carbonate, malic acid, potassium citrate, sodium phosphate, lactic acid, gluconic acid, tartaric acid, fumaric acid, diethanolamine, monoethanolamine, sodium carbonate, sodium bicarbonate, triethanolamine, or any combination thereof. The stable aqueous parenteral solution may comprise one or more pH adjusting agents in an amount to provide pH of the solution between about 8 and about 9, for example about 8.5.

Examples of suitable pharmaceuticcally acceptable isotonicity adjusting agents may include, but not limited to, sodium chloride, potassium chloride, calcium chloride, magnesium chloride, glucose, sucrose, dextrose, mannitol, glycerol, or any combination thereof.

The stable aqueous parenteral solution of the present invention may comprise sodium chloride in a concentration of between about 1 mg/mL and about 10 mg/mL, for example, about 3 mg/mL, about 5 mg/mL or about 7 mg/mL.

In another embodiment, the stable parenteral solution may comprise about 10 mg/mL of meloxicam, about 45 mg/mL of polyvinylpyrrolidone, about 10 mg/mL of polysorbate 80, one or more pH adjusting agents, for example, NaOH and/or NCl in a quantity sufficient to adjust pH of the solution about 8.5 and quantity sufficient water.

In another embodiment, the stable parenteral solution may comprise about 10 mg/mL of meloxicam about 100 mg/mL of polyvinylpyrrolidone, one or more pH adjusting agents, for exa pie, NaOH and/or HCl in a quantity sufficient to adjust pH of the solution about 8.5 and quantity sufficient water.

In another embodiment, the stable parenteral solution may comprise about 15 mg/mL of meloxicam, about 150 mg/mL of polyvinylpyrrolidone about 20 mg/mL of polysorbate 80, about 5 mg/mL of monothioglycerol, one or more pH adjusting agents, for example, NaOH and/or HCl in a quantity sufficient to adjust pH of the solution about 8.5 and quantity sufficient water.

In another embodiment, the stable parenteral solution may comprise about 15 mg/mL of meloxicam, about 200 mg/mL of polyvinylpyrrolidone, one or more pH adjusting agents, for example, NaOH and/or HCl in a quantity sufficient to adjust pH of the solution about 8.5 and quantity sufficient water.

In one embodiment, the present invention provides a stable parenteral solution comprising meloxicam, polyvinylpyrrolidone, polysorbate 80 and water, wherein the solution is substantially free of co-solvent(s), for example, dimethyl sulfoxide, N,N-dimethylformamide, N,N-dimethyl acetamide, benzyl alcohol, N-methyl pyrrolidone or glycerol formal, ethanol, transcutol, glycerol, cremophor (polyethoxylated castor oil), glycofurol, propylene glycol or polyethylene glycol. The term “substantially free of” as sed herein, unless and otherwise specifically mentioned, means total amount of co-solvent(s) (organic and/or inorganic), if present in the solution, is less than 5% v/v of the solution, for example, less than 3% viv, less than 1% v/v, or less than 0.5% v/v of the solution. The solution may be free of any co-solvent.

In another embodiment, the present invention provides a stable parenteral solution comprising meloxicam, polyvinylpyrrolidone, polysorbate 80 and water, wherein the solution does not contain any co-solvent, such as, dimethyl sulfoxide, N,N-dimethylformamide, N,N-dimethyl acetamide, benzyl alcohol, N-methyl pyrrolidone or glycerol formal, ethanol, transcutol, glycerol, cremophor (polyethoxylated castor oil), glycofurol, propylene glycol or polyethylene glycol.

In another embodiment the stable aqueous parenteral solution of the present invention does not contain any toxic and/or irritant ingredient, for example, any cyclodextrin derivative (HPBCD (hydroxypropyl-β-cyclodextrin), HPACD (hydroxypropyl-α-cyclodextrin), HPGCD (hydroxypropyl-γ-cyclodextrin), SBECD (sulfobutylether-β-cyclodextrin) etc.), transcutol, cremophor (polyethoxylated castor oil), glycofurol, propylene glycol and/or polyethylene glycol.

In another embodiment, the stable aqueous parenteral solution of the present invention does not contain meglumine.

In another embodiment, the present invention provides a stable parenteral solution comprising meloxicam and water, wherein the solution is not for any other route of administration except for the parenteral route of administration; for example, solution is not for the administration via ophthalmic route, otic route, topical route (application on skin) or oral route. The stable parenteral solution comprising meloxicam and water is not an eye drop solution and/or ear drop solution.

In one embodiment, the present invention provides stable, aqueous, parenteral solution comprising meloxicam and polyvinylpyrrolidone, wherein the solution is not for the preparation of solid dispersion for oral administration.

In another embodiment the stable aqueous parenteral solution of the present invention does not contain any local anesthetic, for example, lidocaine, prilocaine tetracaine, bupivacaine, mepivacaine and/or xylocaine.

In another embodiment the present invention provides a method of treating acute pain, osteoarthritis, rheumatoid arthritis and juvenile rheumatoid arthritis pauciarticular and polyarticular course, the method comprising administering to a human being in need thereof, the parenteral meloxicam solution of the present invention, via parenteral route. The parenteral dose of meloxicam may range from about 7.5 mg to about 60 mg, for example 10 mg, 15 mg, 20 mg, 25 mg, 30 mg, 35 mg, 40 mg, 45 mg, 50 mg or 55 mg. The treatment of osteoarthritis, rheumatoid arthritis and juvenile rheumatoid arthritis involves relief of the signs and symptoms. The treatment of juvenile rheumatoid arthritis involves relief of the signs and symptoms of pauciarticular or polyarticular course Juvenile Rheumatoid Arthritis in patients 2 years of age and older.

In another embodiment, the present invention provides a method of treating acute pain, the method comprising administering to a human being in need thereof, the parenteral meloxicam solution of the present invention, via intravenous route, for example, intravenous bolus injection.

In another embodiment, the present invention provides a method of treating osteoarthritis, rheumatoid arthritis and juvenile rheumatoid arthritis, the method comprising administering to a human being in need thereof, the parenteral meloxicam solution of the present invention, via subcutaneous route.

In another embodiment, the present invention provides a method of treating pain, the method comprising administering to a human being in need thereof, the parenteral meloxicam solution of the present invention, wherein a need for administration of concomitant opioid analgesic is limited or reduced or absent.

In another embodiment, the present invention provides a process for preparing a stable parenteral solution comprising meloxicam, polyvinylpyrrolidone, polysorbate 80 and water. The process includes steps: (a) adding polyvinylpyrrolidone and polysorbate 80 into water to form a clear solution, (b) adding meloxicam into the solution prepared in step (a), and (c) adding pH adjusting agent like NaOH/HCl to have pH of the solution between about 8 and about 9. Additionally, the process may include adding an appropriate amount of NaCl at the step (a). The prepared solution may be subjected to a terminal sterilization process.

In a other embodiment, the physical and chemical stability of the parenteral solution of the present invention was studied at 25° C. temperature and 60% relative humidity (% RH) as well as at 40° C. temperature and 75% RH.

The solution comprising therapeutically effective amount of meloxicam of the invention may provide value of Tₘₐₓ less than 5 hours, for example, less than 4 hours, less than 3 hours, less than 2 hours, less than 1 hour, less than 45 minutes, less than 30 minutes, less than 20 minutes, less than 10 minutes, less than 5 minutes, less than 3 minutes or less than 1 minute, when the solution is administered via subcutaneous or intramuscular route to a human at a meloxicam dose between of 7.5 mg and 60 mg. The solution comprising therapeutically effective amount of meloxicam of the invention provides immediate availability of the entire dose of meloxicam in the blood when the solution is administered via intravenous route to a human.

The solution comprising meloxicam of the invention may provide value of Cₘₐₓ more than 250 ng/mL, for example, more than 500 mg/mL, more than 1000 ng/mL, more than 1500 ng/mL, more than 2000 ng/mL, more than 2500 ng/mL, more than 3000 ng/mL., more than 3500 ng/mL, more than 4000 ng/mL, more than 4500 ng/mL, more than 5000 ng/mL, more than 5500 ng/mL, more than 6000 ng/mL, more than 6500 ng/mL, more than 7000 ng/mL, more than 7500 ng/mL, more than 8000 ng/mL, more than 8500 ng/mL, more than 9000 ng/mL, more than 9500 ng/mL, more than 10,000 ng/mL, more than 10,500 ng/mL, more than 11,000 ng mL, more than 11,500 ng/mL or more than 12,000 ng/mL, when the solution administered via parenteral route to a human at a meloxicam dose of between 7.5 mg and 60 mg.

The solution comprising meloxicam of the invention may provide value of AUCₗₐₛₜ more than 10 μg*h/mL, more than 20 μg*h/mL, more than 30 μg*h/mL, more than 40 μg*h/mL, more than 50 μg*h/mL, more than 60 μg*h/mL, more than 70 μg*h/mL, more than 80 μg*h/mL, more than 90 μg*h/mL, more than 100 μg*h/mL, more than 110 μg*h/mL, more than 120 μg*h/mL, more than 130 μg*h/mL, more than 140 μg*h/mL, more than 150 μg*h/mL, more than 160 μg*h/mL, more than 170 μg*h/mL, more than 180 μg*h/mL, more than 190 μg*h/mL, more than 200 μg*h/mL, more than 210 μg*h/mL, more than 220 μg*h/mL, more than 230 μg*h/mL, more than 240 μg*h/mL or more than 250 μg*h/mL, when the solution is administered via parenteral route to a human at a meloxicam dose of between 7.5 mg and 60 mg.

The present invention is illustrated by the following examples which are provided to be exemplary of the invention and do not limit the scone of the invention.

### EXAMPLES

### Example 1

Quantities of Ingredients 1 through 5 are in mg.

| Sr. | | |
|---|---|---|
| No. | Ingredients | Quantity |
| 1 | Meloxicam | 1000 |
| 2 | Polyvinylpyrrolidone | 4500 |
| | K12 | |
| 3 | Polysorbate 80 | 1000 |
| 4 | Sodium chloride | 200 |
| 5 | Glycine | 500 |
| 6 | Hydrochloric acid | q.s. to pH 8.5 |
| 7 | Sodium hydroxide | q.s. to pH 8.5 |
| 8 | Water for Injection | q.s. to 100 mL |
| | (WFI) | |

Process for Example 1
Water for injection was kept under nitogen sparging until dissolved oxygen less than 2 mg/L.
2. Accurately weighed polyvinylpyrrolidone K12, polysorbate 80, sodium chloride and glycine were added into 80 mL of water for injection, stirred and dissolved completely at controlled room temperature.
3. Meloxicam was added to the above prepared solution with moderate stirring at controlled room temperature.
4. Then pH of the solution was adjusted to 8.5 using 5% w/v NaOH and/or 1N HCl and the volume was made up to 100 mL using water for injection.
5. The solution: was filtered through a 0.22μ filter and filled into a glass vial.

Stability Data for Example 1

The meloxicam solution obtained at Example 1 was tested for its physical and chemical stability at various time points and storage conditions, and suits are reported in the Tables 1A and 1B below.

TABLE 1A

| | | One month | Nine months | One month | Nine months |
|---|---|---|---|---|---|
| | | 25 ± 2° C. | 25 ± 2° C. | 40 ± 2° C. | 40 ± 2° C. |
| Condition | Initial | 60 ± 5% RH | 60 ± 5% RH | 75 ± 5% RH | 75 ± 5% RH |
| Description | Clear | Clear | Clear | Clear | Clear |
| | yellowish | yellowish | yellowish | yellowish | yellowish |
| | liquid | liquid | liquid | liquid | liquid |
| pH | 8.60 | 8.40 | 8.14 | 8.40 | 8.34 |
| Osmolality (mOsm) | 298 | 299 | 302 | 301 | 302 |
| Absorbance | 3.06 | 3.58 | — | 3.56 | — |
| Transmittance (%) | 99.40 | 99.40 | 99.60 | 99.4 | 98.60 |
| Viscosity (cps) | 1.38 | 1.42 | 2.11 | 1.44 | 1.45 |
| Assay (By HPLC) % | 100.00 | 102.70 | 99.50 | 101.9 | 100.50 |
| Total impurities (%) | Nil | Nil | Nil | Nil | 0.10 |

TABLE 1B

| | | | 25 ± 2° C./ | 40 ± 2° C./ |
|---|---|---|---|---|
| | | | 60 ± 5% RH | 75 ± 5% RH |
| Type of Impurity | | Initial | 12 Months | 6 Months |
| HPLC | Impurity B | Below | Below | 0.08 |
| method (at | | Detectable | Quantification | |
| wavelength | | Limit | Limit | |
| 260 nm) | 5N | Not | Not | Not |
| | Methylthiazol- | detected | detected | detected |
| | 2-yl oxalic | | | |
| | acid | | | |
| | Single maximum | Below | Not | Below |
| | unknown impurity | Detectable | detected | Quantification |
| | | Limit | | Limit |
| HPLC | Impurity A | Not | Not | Not |
| method (at | | detected | detected | detected |
| wavelength | N-Methyl | Not | Not | Not |
| 350 nm) | Meloxicam | detected | detected | detected |
| | Single maximum | Below | Not | Below |
| | unknown impurity | Detectable | detected | Quantification |
| | | Limit | | Limit |

| | | | |
|---|---|---|---|
| Total Impurities | Below | Below | 0.1 |
| | Detectable | Quantification | |
| | Limit | Limit | |

### Example 2

Quantities of Ingredients 1 through 4 are in mg.

| Sr. No. | Ingredients | Quantity |
|---|---|---|
| 1 | Meloxicam | 1500 |
| 2 | Polyvinylpyrrolidone | 6750 |
| | K12 | |
| 3 | Polysorbate 80 | 1000 |
| 4 | Glycine | 500 |
| 5 | Hydrochloric acid | q.s. to pH 8.5 |
| 6 | Sodium hydroxide | q.s. to pH 8.5 |
| 7 | Water for Injection | q.s. to 100 mL |
| | (WFI) | |

Process for Example 2:
1. Water for injection was kept under nitrogen sparging until dissolved oxygen less than 2 mg/L.
2. Accurately weighed polyvinylpyrrolidone K12, polysorbate 80 and glycine were added into 80 mL of water for injection, stirred and dissolved completely at controlled room temperature.
3. Meloxicam was added to the above prepared solution with moderate stirring at controlled room temperature.
4. Then pH of the solution was adjusted to 8.5 using 5% w/v NaOH and/or 1N HCl and the volume was made up to 100 mL using water for injection.
5. The solution was filtered through a 0.22μ filter and filled into a glass vial.

### Example 3

Quantities of Ingredients 1 through 5 are in mg.

| Sr. No. | Ingredients | Quantity |
|---|---|---|
| 1 | Meloxicam | 3000 |
| 2 | Polyvinylpyrrolidone | 15000 |
| | K12 | |
| 3 | Polysorbate 80 | 1500 |
| 4 | Glycine | 500 |
| 5 | Monothioglycerol | 500 |
| 6 | Hydrochloric acid | q.s. to pH 8.5 |
| 7 | Sodium hydroxide | q.s. to pH 8.5 |
| 8 | Water for Injection | q.s. to 100 mL |
| | (WFI) | |

Process for Example 3:
1. Water for injection was kept under nitrogen sparging until dissolved oxygen less than 2 mg/L.
2. Accurately weighed polyvinylpyrrolidone K12, polysorbate 80, glycine and monothioglycerol were added into 80 mL of water for injection, stirred and dissolved completely at controlled room temperature.
3. Meloxicam was added to the above prepared solution with moderate stirring at controlled room temperature.
4. Then pH of the solution was adjusted to 8.5 using 5%, w/y NaOH and/or 1N HCl and the volume was made up to 100mL using water for injection.
5. The solution was filtered through a 0.22μ filter and filled into a glass vial/

Stability Data for Example 3

The meloxicam solution obtained at Example 3 was tested for its physical and chemical stability, at initial point, and results are reported in the Tables 3A and 3B below.

TABLE 3A

| Condition | Initial |
|---|---|
| Description | Clear yellowish liquid |
| pH | 8.7 |
| Osmolality (mOsm) | 578 |
| Transmittance (%) | 100.0 |
| Viscosity (cps) | 3.2 |
| Assay (By HPLC) % | 101.8 |
| Total impurities (%) | Below Quantification limit |

TABLE 3B

| Type of Impurity | | Initial |
|---|---|---|
| HPLC method (at | Impurity B | Below |
| wavelength 260 nm) | | Quantification Limit |
| | 5N Methylthiazol-2-yl | Not detected |
| | oxalic acid | |
| | Meloxicam diazenyl | Not detected |
| | sulfonyl Impurity | |
| | Single maximum | Not detected |
| | unknown impurity | |
| HPLC method (at | Impurity A | Not detected |
| wavelength 350 nm) | N-Methyl Meloxicam | Not detected |
| | Single maximum | Below |
| | unknown impurity | Quantification Limit |

| | |
|---|---|
| Total Impurities | Below |
| | Quantification Limit |

### Example 4

Quantities of Ingredients 1 through 4 are in mg.

| Sr. | | |
|---|---|---|
| No. | Ingredients | Quantity |
| 1 | Meloxicam | 3000 |
| 2 | Polyvinylpyrrolidone | 15000 |
| | K12 | |
| 3 | Polysorbate 80 | 1000 |
| 4 | Glycine | 500 |
| 5 | Hydrochloric acid | q.s. to pH 8.5 |
| 6 | Sodium hydroxide | q.s. to pH 8.5 |
| 7 | Water for Injection | q.s. to 100 mL |
| | (WFI) | |

Process for Example 4:
Water for injection was kept under nitrogen sparging until dissolved oxygen less than 2 mg/L.
2. Accurately weighed polyvinylpyrrolidone K12, polysorbate 80, and glycine were added into 80 mL of water for injection, stirred and dissolved completely at controlled room temperature.
3. Meloxicam was added to the above prepared solution with moderate stirring at controlled room temperature.
4. Then pH of the solution was adjusted to 8.5 using 5% w/v NaOH and/or 1N HCl and the volume was made up to 100 mL using water for injection.
5. The solution was filtered through a 0.22μ filter and filled into a glass vial.

Stability data for Example 4

The meloxicam solution obtained at Example 4 was tested for its physical and chemical stability, at various time points and storage conditions, and results are reported in the Tables 4A and 4B below.

TABLE 4A

| | | One month | Three months | One month | Three months |
|---|---|---|---|---|---|
| | | 25 ± 2° C. | 25 ± 2° C. | 40 ± 2° C. | 40 ± 2° C. |
| Condition | Initial | 60 ± 5% RH | 60 ± 5% RH | 75 ± 5% RH | 75 ± 5% RH |
| Description | Clear | Clear | Clear | Clear | Clear |
| | yellowish | yellowish | yellowish | yellowish | yellowish |
| | liquid | liquid | liquid | liquid | liquid |
| pH | 8.5 | 8.2 | 8.4 | 8.2 | 8.3 |
| Osmolality (mOsm) | 498 | 493 | 499 | 497 | 495 |
| Transmittance (%) | 99.3 | 98.8 | 99.8 | 99.0 | 99.1 |
| Viscosity (cps) | 3.7 | 3.1 | 3.2 | 3.1 | 3.2 |
| Assay (By HPLC) % | 98.9 | 99.7 | 101.3 | 98.6 | 100.8 |
| Total impurities (%) | 0.4 | 0.5 | 0.4 | 0.4 | 0.5 |

TABLE 4B

| | | | 25 ± 2° C./ | 40 ± 2° C./ |
|---|---|---|---|---|
| | | | 60 ± 5% RH | 75 ± 5% RH |
| Type of Impurity | | Initial | 3 Months | 3 Months |
| HPLC method (at | Impurity B | Below | 0.10 | 0.14 |
| wavelength 260 nm) | | Quantification | | |
| | | Limit | | |
| | 5N | 0.13 | Below | Below |
| | Methylthiazol- | | Quantification | Quantification |
| | 2-yl oxalic acid | | Limit | Limit |
| | Meloxicam diazenyl | 0.24 | 0.34 | 0.37 |
| | sulfonyl Impurity | | | |
| | Single maximum | Below | Not | Below |
| | unknown impurity | Quantification | detected | Quantification |
| | | Limit | | Limit |
| HPLC method (at | Impurity A | Not | Not | Not |
| wavelength 350 nm) | | detected | detected | detected |
| | N-Methyl | Below | Not | Not |
| | Meloxicam | Detectable | detected | detected |
| | | Limit | | |
| | Single maximum | Not | Below | Below |
| | unknown impurity | detected | Quantification | Quantification |
| | | | Limit | Limit |

| | | | |
|---|---|---|---|
| Total Impurities | 0.4 | 0.4 | 0.5 |

White the present invention has been described in terms of its specific embodiments, certain modifications and equivalents will be apparent to those skilled in the art and are intended to be included within the scope of the present invention.

A parenteral solution comprising meloxicam, polyvinylpyrrolidone and water, wherein the solution does not contain any cyclodextrin derivative or meglumine.

2. The parenteral solution according to claim 1, wherein the solution does not contain any co-solvent.

3. The parenteral solution according to claim 1, wherein the solution further comprises one or more surfactant.

4. The parenteral solution according to claim 3, wherein the surfactant is polysorbate 80.

5. The parenteral solution according to claim 1, wherein the solution further comprises one or more pH adjusting agents.

6. The parenteral solution according to claim 1, wherein the meloxicam is present in a concentration of between about 5 mg/mL and about 50 mg/mL.

7. The parenteral solution according to claim 1, wherein the polyvinylpyrrolidone is present in a concentration of between about 10 mg/mL and about 100 mg/mL.

8. The parenteral solution according to claim 4, wherein the polysorbate 80 is present in a concentration of between about 5 mg/mL and about 20 mg/mL.

9. The parenteral solution according to claim 1, wherein the solution has a pH of between about 7 and about 10.

10. The parenteral solution according to claim 1, wherein the solution has a viscosity value of between about 1 cP and 5 cP.

11. The parenteral solution according to claim 1, wherein the solution has an osmolality value of between about 200 mOsm and about 600 mOsm.

12. The parenteral solution according to claim 1, wherein the solution does not contain total impurities more than 3% after storage for 6 months at 25±2° C. temperature and 60±5% RH.

13. The parenteral solution according to claim 1, wherein the solution does not form any precipitate after storage for 6 months at 25±2° C. temperature and 60±5% RH.

14. The parenteral solution according to claim 1, wherein the solution retains at least 95% of the meloxicam (% assay) after storage for 6 months at 25±2° C. temperature and 60±5% RH.

15. The parenteral solution according to claim 1, wherein the solution does not contain 2-((diazenyisulfonyl)-N-methylaniline oxide impurity more than 1% after storage for 6 months at 25±2° C. temperature and 60±5% RH.

16. A parenteral solution comprising meloxicam, polyvinylpyrrolidone and water, wherein the solution does not contain 2-Amino-5-methyl-thiazole (impurity B) more than 0.5% after storage for 6 months at 25±2° C. temperature and 60±5% RH.

17. A method of treating pain and/or arthritis, the method comprising administering to a human being in need thereof, a solution, via parenteral route, wherein the solution comprising meloxicam, polyvinylpyrrolidone, polysorbate 80, sodium chloride, one or more pH adjusting agents and water.

18. The method according to claim 17, wherein the pain is acute pain.

19. The method according to claim 17, wherein the arthritis is osteoarthritis, rheumatoid arthritis or juvenile pauciarticular and polyarticular rheumatoid arthritis.

20. The method according to claim 17, wherein the method comprises administering the solution via parenteral route, wherein the meloxicam is present from about 7.5 mg to about 60 mg.
